(19) United States
(12) Patent Application Publication
(10) Pub. No.: US 2006/0070623 A1
(43) Pub. Date: Apr. 6, 2006

(54) Method and apparatus for determining a bodily characteristic or condition

(76) Inventors: Malcolm Howard Wilkinson, Forest Hill (AU); Clive Andrew Ramsden, Cheltenham (AU); Philip John Berger, Carlton (AU)

Correspondence Address: PARSONS HSUE & DE RUNTZ LLP, 595 MARKET STREET, SUITE 1900, SAN FRANCISCO, CA, 94105, US

(21) Appl. No.: 11/124,326

(22) Filed: May 6, 2005

Related U.S. Application Data
(63) Continuation-in-part of application No. 10/272,494, filed on Oct. 15, 2002.
Application No. 10/272,494 is a continuation of application No. PCT/AU01/00465, filed on Apr. 20, 2001.

(30) Foreign Application Priority Data
Apr. 10, 2001 (AU) AU PR4333
Apr. 20, 2000 (AU) AU PQ7040

(51) Int. Cl. A61M 16/00; A61B 5/08

(52) U.S. Cl. 128/204.23; 600/529

(57) ABSTRACT

The present invention relates to a method of determining at least one bodily characteristic or condition, such as that of an animal, such as a human, for example. According to an aspect of the invention, the method relates to determining at least one bodily characteristic of a lung or an airway, merely by way of example, by introducing at least one sound to at least one first bodily location, and recording at least one sound from at least one second bodily location. The present invention also relates to an apparatus capable of such determination.

### CROSS-REFERENCE TO RELATED APPLICATIONS

The present application is a continuation-in-part of U.S. application Ser. No. 10/272,494 of Wilkinson et al., filed on Oct. 15, 2002, which is a continuation of Patent Cooperation Treaty Application No. PCT/AU01/00465, filed on Apr. 20, 2001, which claims priority to Australian Provisional Application Nos. AU PQ7040 and AU PR4333, filed on Apr. 20, 2000 and Apr. 10, 2001, respectively. The parent application, U.S. application Ser. No. 10/272,494, was published as U.S. Patent Application Publication No. 2003/0120182 A1. The present application is also related to Australian Application Nos. AU 2001252025 and 2004222800, filed on Apr. 20, 2001 and Oct. 4, 2004, respectively. Each of the foregoing applications, provisional applications, and publications, is hereby incorporated herein, in its entirety, by this reference.

The present invention relates to a method of determining a bodily characteristic or condition. The invention further relates to an apparatus capable of such determination.

### BACKGROUND

Non-invasive determination of the condition of biological tissues is useful in particular where the patient is unable to co-operate or the tissue is inaccessible for easy monitoring.

Techniques presently used in determining the characteristics of biological tissues include x-rays, magnetic resonance imaging (MRI) and radio-isotopic imaging. These are generally expensive and involve some degree of risk which is usually associated with the use of X-rays, radioactive materials or gamma-ray emission. Furthermore, these techniques are generally complicated and require equipment which is bulky and expensive to install and, in most cases, cannot be taken to the bedside to assess biological tissues in patients whose illness prevents them being moved.

Sound waves, particularly in the ultra-sound range have been used to monitor and observe the condition of patients or of selected tissues, such as the placenta or fetus. However, the process requires sophisticated and sometimes expensive technology and cannot be used in tissues in which there is a substantial quantity of gas, such as the lung.

Every year in Australia about 5000 newborn infants require a period of intensive care (ANZNN Annual Report, 1996-1997). Respiratory failure is the most common problem requiring support and is usually treated with a period of mechanical ventilation. Over the last decade the mortality of infants suffering respiratory failure has shown an impressive decline, attributable at least in part to improved techniques used in mechanical ventilation, and the introduction of surfactant replacement therapy (Jobe, 1993). The vast majority of infants now survive initial acute respiratory illness, but lung injury associated with mechanical ventilation causes many infants to develop ‘chronic lung disease’. Chronic lung disease is characterised by persisting inflammatory and fibrotic changes, and causes over 90% of surviving infants born at less than 28 weeks gestation, and 30% of those of 28-31 weeks gestation, to be dependent on supplementary oxygen at 28 days of age. Of these, over half still require supplementary oxygen when they have reached a post-menstrual age of 36 weeks gestation (ANZNN Annual report, 1996-1997). Assistance with continuous positive airway pressure (CPAP) or artificial ventilation is also commonly required.

Historically, barotrauma and oxygen toxicity have been considered to be the primary culprits in the aetiology of chronic lung disease (Northway et al, 1967; Taghizadeh & Reynolds, 1976). However, trials of new strategies in mechanical ventilation which were expected to reduce barotrauma and/or exposure to oxygen have often had disappointingly little impact on the incidence of chronic lung disease (HIFI Study Group, 1989; Bernstein et al, 1996; Baumer, 2000). Comparison of strategies of conventional mechanical ventilation in animals (Dreyfuss et al, 1985) have indicated that high lung volumes may be more damaging than high intrapulmonary pressures, and has led to the concept of ‘volutrauma’ due to over-inflation of the lung. At the same time, experience with high frequency oscillatory ventilation (HFOV) has indicated that avoidance of under-inflation may be equally important. HFOV offers the potential to reduce lung injury by employing exceptionally small tidal volumes which are delivered at a very high frequency. However, this technique fails to confer benefit, if the average lung volume is low (HIFI Study Group, 1989), yet it appears to be successful if a normal volume is maintained (McCulloch et al, 1988; Gerstmann et al, 1996). This highlights the importance of keeping the atelectasis-prone lung ‘open’ (Froese, 1989). Evidence of this kind has led to the concept that a ‘safe window’ of lung volume exists within which the likelihood of lung injury can be minimised. The key to preventing lung injury may lie in maintaining lung volume within that safe window thereby avoiding either repetitive over-inflation or sustained atelectasis. (See FIG. 1.)

Attempts to maintain an optimal lung volume in the clinical setting are frustrated by a lack of suitable methods by which the degree of lung inflation can be monitored. In current practice, evaluation of oxygen requirements and radiological examination of the lungs are the principal techniques employed. However, oxygen requirements may be influenced by factors other than lung volume (for example intra- or extracardiac right to left shunting), and the hazards of radiation exposure prevent radiological examination being performed with the frequency required.

Monitoring of infants during mechanical ventilation has been significantly improved over the last decade by the incorporation of a pneumotachograph or hot-wire anemometer into the design of many neonatal ventilators. Although this provides a valuable tool for monitoring tidal volume and compliance, it gives only the most indirect indication (from the shape of the pressure-volume curve) of whether that tidal volume is being delivered in a setting of under-inflation, optimal inflation, or over-inflation. Furthermore, while absolute lung gas volume can be measured using ‘gold-standard’ techniques of Nitrogen (N₂) washout or Helium (He) dilution, these are impractical for routine clinical use.

Even when lung volume is maintained in the “safe window”, changes in the lung condition may manifest due to the general damaged or underdeveloped condition of the lung. Fluid and blood may accumulate in the lung, posing additional threats to the patient. Evaluation with a stethoscope of audible sounds which originate from within the lung (breath sounds) or are introduced into the lung (by percussion, or as vocal sounds) forms an essential part of any routine medical examination. However, in the sick newborn, the infant's small size, inability to co-operate and the presence of background noise greatly limits the value of such techniques.

Whilst determining and monitoring lung condition in newborn babies is difficult, determining lung condition in adults can be equally challenging, particularly if a patient is unconscious or unable to cooperate. This places a further limitation on the presently available techniques for monitoring lung condition. Therefore, a clear need exists for a simple, non-invasive and convenient method by which the condition of the lung can be closely monitored in the clinical setting. Similarly, there is a need for a simple, non-invasive and convenient method of determining the condition of other biological tissues which may be prone to changes in their characteristics, through pathology or otherwise.

Further development of methods and apparatus or systems for determining a bodily characteristic or condition is desirable, whether in terms of some degree of progress towards the achievement of any of the needs or desires just described, or otherwise.

### SUMMARY

In an aspect of the present invention there is provided a method of determining characteristics of biological tissue in situ, including:

introducing a sound to the tissue at a first position;

detecting the sound at another position spaced from the first position after it has travelled through the tissue;

calculating the velocity and attenuation of sound that has travelled through the tissue from the first position to another position; and

correlating the velocity and attenuation of the detected sound to characteristics of the biological tissue.

In another aspect of the present invention there is provided a method of determining characteristics of tissue of the respiratory system in situ, said method including:

introducing an audible sound to the tissue at a first position;

detecting the sound at another position spaced from the first position after it has travelled through the tissue;

calculating the velocity and attenuation of sound that has travelled through the tissue from the first position to another position; and

correlating the velocity and attenuation of the detected sound to characteristics of the tissue of the respiratory system.

In another aspect of the present invention there is provided an apparatus for determining characteristics of tissue of the respiratory system, the apparatus including:

a sound generating device which generates an audible sound;

a recording device which records the sound after it has travelled from one position of the respiratory system tissue, through the tissue and to another position of the tissue;

an analysis device which calculates the velocity with which the sound travels through the tissue, and its attenuation, and which can preferably perform spectral analysis on the data recorded.

In another aspect of the present invention, there is provided a method of determining a state of the upper airways in a respiratory tract in a patient in situ, said method including:

introducing an audible sound at a first position in the upper airways;

detecting the sound after it has travelled through the upper airways at another position spaced from the first position;

calculating the velocity and attenuation of the sound that has travelled through the upper airways from the first position to another position; and

correlating the velocity and attenuation of the sound to a state of the upper airways.

This method is particularly useful for monitoring for sleep apnea.

According to an aspect of the present invention, a method may be directed to determining placement of a structure within an airway. Such a method may be used to determine a placement, such as a correct or incorrect placement, for example, of a medical device, such as an endo-tracheal tube, for example, within an airway. This may be useful in a medical application in which placement of a medical device, and/or the monitoring thereof, is contemplated, for example. Such a method may be used to determine a placement, such as a location, for example, of an undesirable structure, such as an obstruction, for example, within an airway. This may be useful with respect to location of such a structure, and/or the monitoring thereof, and/or the removal thereof, for example. Other applications of such a method will be understood. Such a method may comprise introducing at least one audible sound to at least one first bodily location associated with an airway, such as an upper airway, for example, the at least one audible sound sufficient to travel through at least a portion of the body to produce at least one responsive sound; receiving the at least one responsive sound from at least one second bodily location, such as a location that is spaced from the first bodily location, for example; determining an attenuation associated with the at least one responsive sound; and determining a placement associated with a structure within the airway, such as via correlating the attenuation to a placement of the structure, for example.

In an aspect of the present invention there is provided a method of monitoring lung condition in situ said method comprising:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

correlating the attenuation, sound velocity and velocity dispersion to lung condition.

Previous work shows that measurement of sound velocity alone may provide a technique for assessing lung density and gives an insight into the degree of lung inflation. However, no attempt has been made to evaluate the potential utility of the measurement of sound velocity and attenuation as a clinical tool.

In yet another aspect of the present invention there is provided a method of measuring lung inflation, said method including:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

correlating changes in sound velocity and attenuation with lung volume and inflation.

In yet another aspect of the present invention, there is provided a method of predicting chronic lung disease in infants said method including:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

comparing the measured sound velocity and attenuation with that of a normal lung in the absence of chronic lung disease.

In yet another aspect of the invention there is provided a method of diagnosing lung disease, said method including measuring lung volume including:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

correlating sound velocity and attenuation with lung density and comparing the density of the lung being diagnosed with the density of a normal lung to determine if the lung being diagnosed is diseased.

According to another aspect of the invention, a method may be directed to diagnosing lung disease. Such a method may comprise:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

estimating lung density from sound velocity and attenuation and comparing the density of the lung being diagnosed with the density of a normal lung to determine if the lung being diagnosed is diseased.

Such a method may further comprise the measuring of lung volume.

In yet another aspect of the present invention, there is provided a method of preventing lung injury, said method including monitoring lung condition by:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax;

correlating the sound velocity and attenuation with lung volume; and

maintaining a lung volume at an optimal volume such that the lung is substantially free of atelectasis or over-inflation (volutrauma).

In yet another aspect of the present invention, there is provided an apparatus for monitoring lung condition, said apparatus including:

a sound generating means to generate an audible sound transthoracically so that the sound travels from one side of the thorax, through the lung, to another side of the thorax;

a recording means to record the sound after it has travelled from one side of the thorax, through and across the lung, to the other side of the thorax;

an analysis device which calculates the attenuation and velocity with which the sound travels from one side of the thorax, through and across the lung, to the other side of the thorax, and which can preferably perform spectral analysis on the data recorded.

According to another aspect of the present invention, an apparatus may be provided for monitoring lung condition. Such an apparatus may comprise:

a sound generating means, or a sound generator, for generating an audible sound transthoracically so that the sound travels from one side of the thorax, through the lung, to another side of the thorax;

a recording means, or a recorder, for recording the sound after it has travelled from one side of the thorax, through and across the lung, to the other side of the thorax;

an analysis device which calculates the velocity with which the sound travels from one side of the thorax, through and across the lung, to the other side of the thorax, and a degree of attenuation of the sound during transit through the lung. The analysis device of such an apparatus may be capable of performing a spectral analysis concerning the data recorded.

These and various other aspects, features and embodiments of the present invention are further described herein.

### DESCRIPTION OF DRAWINGS

A detailed description of various aspects, features and embodiments of the present invention is provided herein with reference to the accompanying drawings, which are briefly described below. The drawings are illustrative and are not necessarily drawn to scale. The drawings illustrate various aspects or features of the present invention and may illustrate one or more embodiment(s) or example(s) of the present invention in whole or in part. A reference numeral, letter, and/or symbol that is used in one drawing to refer to a particular element or feature may be used in another drawing to refer to a like element or feature.

FIG. 1 shows a pressure-volume curve of a moderately diseased lung illustrating two hazardous regions of lung volume, and indicating an optimal “safe” window there between (from Froese, 1997).

FIG. 2 includes a panel A, which shows sound pressure level (SPL) (dB) and sound velocity (m/s) versus frequency (Hz) for pooled results taken from 6 adult subjects during breath-holds at residual volume (RV), functional residual capacity (FRC) and total lung capacity (TLC); and a panel B, which shows results from an infant of 26 weeks gestation with healthy lungs, each data point representing the pooled mean±S.E. of 5 measurements. The results were obtained from a reference position in the adult with the transducer at the 2nd right intercostal space on the anterior chest wall and in the newborn over the right upper chest. In both adult and infant, the microphone was placed on the opposite wall of the chest directly in line with the transducer.

FIG. 3 illustrates the relationship between sound velocity and the volumetric fraction of tissue and the average lung density.

FIG. 4(a) illustrates an electric circuit which models the acoustic characteristics of the thorax. FIG. 4(b) illustrates (1) large, (2) moderate and (3) small acoustic losses as measured using the electric circuit model and which represents the output SPL as would be measured at a chest microphone when the input SPL is 105 dB.

FIG. 5(a) shows the SPL measured at a chest microphone, recorded before (pre) and after (post) administration of surfactant in 3 preterm infants, wherein the sound level produced by the transducer was 105 dB (Sheridan 2000). FIG. 5(b) shows the electric model simulation of FIG. 5(a), demonstrating the change in the SPL measured at the chest wall following a 3-fold increase in lung gas compliance, wherein the sound level produced by the transducer was, again, 105 dB.

FIG. 6 shows the relationship between frequency and the attenuation coefficient, α, plotted with tissue fraction, h, as a parameter.

Each of FIG. 7(a) and FIG. 7(b), independently, is an illustration of an apparatus or system configured according to an embodiment of the present invention.

FIG. 8 is a schematic illustration of an apparatus or system according to an embodiment of the present invention. Such an apparatus or system may used in connection with a human infant, as shown, merely by way of example.

FIG. 9 is a schematic illustration of a portion of the apparatus or system shown in FIG. 8.

### DESCRIPTION

In an aspect of the present invention there is provided a method of determining characteristics of biological tissue in situ, including:

introducing a sound to the tissue at a first position;

detecting the sound at another position spaced from the first position after it has travelled through the tissue;

calculating the velocity and attenuation of sound that has travelled through the tissue from the first position to another position; and

correlating the velocity and attenuation of the detected sound to characteristics of the biological tissue.

In another aspect of the present invention there is provided a method of determining characteristics of tissue of the respiratory system in situ, said method including:

introducing an audible sound to the tissue at a first position;

detecting the sound at another position spaced from the first position after it has travelled through the tissue;

calculating the velocity and attenuation of sound that has travelled through the tissue from the first position to another position; and

correlating the velocity and attenuation of the detected sound to characteristics of the tissue of the respiratory system.

Characteristics of biological tissues can be determined by measuring the velocity and attenuation of a sound as it propagates through the tissue. This can be achieved by introducing a sound to a particular location or position on the tissue, allowing the sound to propagate through the tissue and measuring the velocity and attenuation with which the sound travels from its source to its destination, wherein the destination includes a receiver which is spatially separated from the sound's source.

Characteristics of the biological tissue may include a feature of the tissue including but limited to its make-up, volume, condition or position in the body.

Biological tissues may include any single tissue or a group of tissues making up an organ or part or region of the body. The tissue may comprise a homogeneous cellular material or it may be a composite structure such as that found in regions of the body including the thorax which for instance can include lung tissue, gas, skeletal tissue and muscle tissue. The tissue may be porous and may comprise a composite structure made up of tissue and gas or has regions of high and low density such as that found in bone tissue.

The tissue may be of the respiratory system. The tissue may be lung tissue or from the upper airway of the respiratory system. The upper airway may include the buccal region extending to the trachea before entering the lungs.

Throughout the description and claims of this specification, the word “comprise” and variations of the word, such as “comprising” and “comprises”, are not intended to exclude other additives, components, integers or steps.

An understanding of the theoretical aspects of sound transmission in tissue is essential for the best use of bio-acoustic data obtained using the present invention.

A unique feature of sound propagation through the lung parenchyma is that the sound velocity is less than that expected for either tissue (1500 ms⁻¹) or air (343 ms⁻¹). This can be explained, in part, by examining the basic relationship between sound velocity v and the physical properties of the lung tissue through which the sound is propagating. This relationship is:

$$v = \frac{1}{\sqrt{ρ C}} , \qquad ( 1 )$$

where ρ is the density and C is the volumetric compliance or inverse volumetric stiffness per unit volume. In determining the velocity of sound in air, substituting an air density of 1.2 kgm⁻³ and an air compliance of 7.14×10⁻⁶ Pa⁻¹ yields a sound velocity in air of 342 ms⁻¹.

Rice (1983) has shown that this relationship also holds for composite porous materials with a closed cell structure which is similar to that of the lung, but where ρ and C are replaced by the tissue's average or composite values. Expressing these values in terms of the volumetric fraction of tissue h and of gas (1−h) and the constituent densities and compliances gives tissue density:
ρ=(1−h)ρ_{g}+hρₜ (2),
and volumetric compliance:
C=(1−h)C_{g}+hCₜ (3),
where ρ, ρ_{g}, ρₜ are the composite, gas and tissue densities respectively and C,C_{g},Cₜ are the composite, gas and tissue volumetric compliances respectively.

Substituting equations (2) and (3) into equation (1) yields an expression which relates sound velocity through a composite structure to the volumetric fraction and the physical properties of both the tissue and gas which compose the material:

$$v = \frac{1}{\sqrt{( ( 1 - h ) {ρ}_{g} + h {ρ}_{t} ) ( ( 1 - h ) {C}_{g} + h {C}_{t} )}} . \qquad ( 4 )$$

It must also be noted that the density of air is approximately 3 orders of magnitude less than that of most tissues and the volumetric compliance of air is some 4 orders of magnitude larger than that of most tissues. This can be used to determine the velocity of sound propagation through the lung for a range of volumetric fractions that are likely to be seen in the lung, (0.05 at TLC to 0.5 to 0.9 for a fully atelectatic or collapsed lung). These velocities can be determined by simplifying equation 4 as follows:

$$v = \frac{1}{\sqrt{h ( 1 - h )}} \frac{1}{\sqrt{{ρ}_{t} {C}_{g}}} . \qquad ( 5 )$$

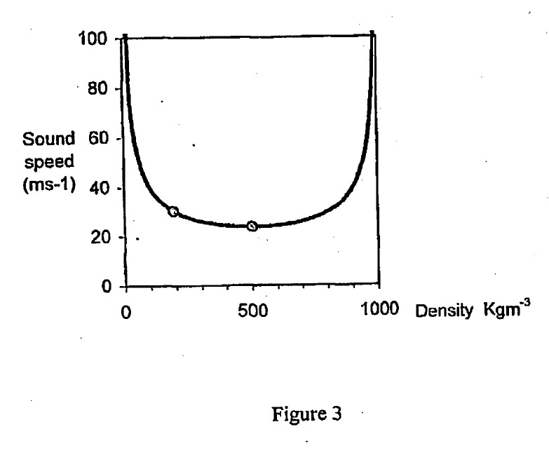

Equation 5, in combination with FIG. 3 illustrates the dependence that sound velocity has on the volumetric fraction of tissue, the volumetric fraction of air, the tissue density and the gas compliance. The tissue, compliance and the gas density play essentially no role in the determination of velocity.

Sound velocity in composite materials is determined in part by the product of the tissue density and the gas compliance. Effectively, the lung parenchyma appears to act like homogeneous mass-loaded air as far as sound propagation is concerned, such that the velocity of sound propagation through the tissue is markedly slower than through air. Substitution of known values for tissue density, ρₜ, and gas compliance, C_{g} in equation 5 gives:

$$v = \frac{11.82}{\sqrt{h ( 1 - h )}} . \qquad ( 6 )$$

Differentiation of v in equation 6 with respect to h determines a minimum value for velocity at h=0.5 where v=23.6 ms⁻¹. For values of h<0.5 the velocity increases with decreasing lung density and conversely for h>0.5 the velocity decreases with decreasing lung density. This is clarified by way of illustration in FIG. 3.

The quadratic properties of equation 6 result in the presence of two values for h for any particular value of measured velocity. These values are:

$$h = 0.5 \pm \sqrt{0.25 - 139.56 / {v}^{2}} . \qquad ( 7 )$$

Therefore, the determination as to whether h is above or below 0.5 must be made on physical grounds or by making paired velocity measurements where h is changed between measurements. The direction of the associated change in velocity (increasing or decreasing) can then be used to indicate whether h is above or below 0.5. Therefore, the volumetric fraction of tissue and gas in the lung and hence lung density can be determined directly from measuring the velocity of sound as it propagates through the tissue.

The sound may be introduced in any non-invasive manner, such as by percussion, or using any mechanical, electrical or other transducer that is capable of generating acoustic sounds. It is preferable that the sound introduced to the tissue possesses properties that allow it to easily be distinguished from environmental noise that may be present. Examples may include a single tone or a sinusoidal wave. In a preferred embodiment of the invention, a pseudo-random noise is produced by an electro-acoustic transducer and introduced into the tissue. The transducer is preferably attached to the surface of the biological tissue through which sound velocities are being measured. It is preferred that the pseudo-random noise signal which is used has characteristics which are similar to a white noise signal, but with mathematical properties which allow its amplitude to be defined at any moment in time. Furthermore, it is preferred that introduction of the pseudo-random noise signal to the tissue occurs in bursts, preferably of 0.1 to 20 seconds duration, and the sounds are produced preferably with frequencies which range from 20 Hz to 25 kHz and at a sound pressure level of between 1 and 100 Pascal.

The sound can then be recorded at a location spaced from the position at which the sound is introduced, preferably on the surface of the biological tissue which is spatially distinct from the location of the transducer, using a sound detection means such as a microphone or a vibration detector, such as an accelerometer, which has a known response, preferably between 20 Hz and 25 kHz. It is preferred that there are at least two of these detectors used to measure the sound, wherein one detector is positioned near a sound-generating acoustic transducer, and another is located at a position spaced from the first position of the tissue being assessed. This enables the sound pressure level, phase, and frequency content of the signal which is produced by the acoustic transducer (the input signal) to be accurately defined before it is detected by the spatially separated second detector. Placement of the second detector is preferably substantially in line with the acoustic transducer and the first detector.

The detector or preferably a microphone output may be amplified using low noise isolation amplifiers and band-pass filtered with cut-off frequencies and roll-off characteristics which depend on the acoustic properties of the tissue which is being assessed. For example, for measurements made on the neonatal lung, the pass band is preferably between 50 Hz and 5 KHz with a roll-off which corresponds to that of a 4^{th} order linear phase filter. These filters remove any very low frequency environmental noise (e.g. below 10 Hz) that can adversely affect the performance of auto-scaling amplifiers into which the filtered signal may be fed.

The amplified output signal from the detector or microphone can then be processed by any means necessary, and a cross-correlation analysis of the input and output signals performed.

The cross-correlation function can be calculated using the output of the microphone which is located in close proximity to the acoustic transducer as the input signal, x(t) and the output of the second microphone located on the other side of the tissue as the output signal, y(t) wherein the cross-correlation function can be calculated as:

$${R}_{xy} ( τ ) = {lim}_{T \to ∞} \frac{1}{T} {\int }_{0}^{T} x ( t ) y ( t + τ ) ⅆ t ,$$

where T is the observation time, and τ is the delay time between x(t) and y(t) at which R_{xy}(τ) is calculated.

It is preferable that the cross-correlation function, which is the impulse response of the system, then undergoes Fast Fourier Transformation so that the signal is transformed into the frequency domain and the transfer function of the tissue can be determined. This transfer function provides a quantitative indication of the characteristics of the tissue, wherein:

(a) the magnitude of the transform provides data relating to the transmission of the sound as it propagates through the tissue as a function of frequency (Rife and Vanderkooy, 1989); and

(b) the phase of the transform (after “unwrapping”) can be used to calculate the phase difference, time delay and velocity of the sound for each frequency that is present in the pseudo-random noise signal which is introduced to the tissue by the acoustic transducer.

Commercially available acoustic hardware and software packages may be used to generate the pseudo-random noise signal, and to perform initial data processing. External noise which is not introduced to the tissue as part of the pseudo-random noise signal is strongly suppressed by the cross-correlation process thereby improving the quality of the measurements made.

A separate analysis of the relative transmission of the sound through the tissue can be used to identify resonant and anti-resonant frequencies of the tissue which is being assessed. Changes in these frequencies can then be used to assess regional differences in tissue topology which may be related to pathology.

Despite numerous experimental investigations (Kraman 1983, Goncharoff et al. 1989, Wodicka and Shannon 1990) of trans-pulmonary sound transmission where the source of sound is placed at the mouth, there has been no theoretical model which described sound transmission through the thorax. The present invention uses a simple model, based on the double wall transmission model that is used in architectural acoustics (Fahy 1985) to describe the sound attenuating effect of double walls separated by a compliant air layer, as is present in the lung.

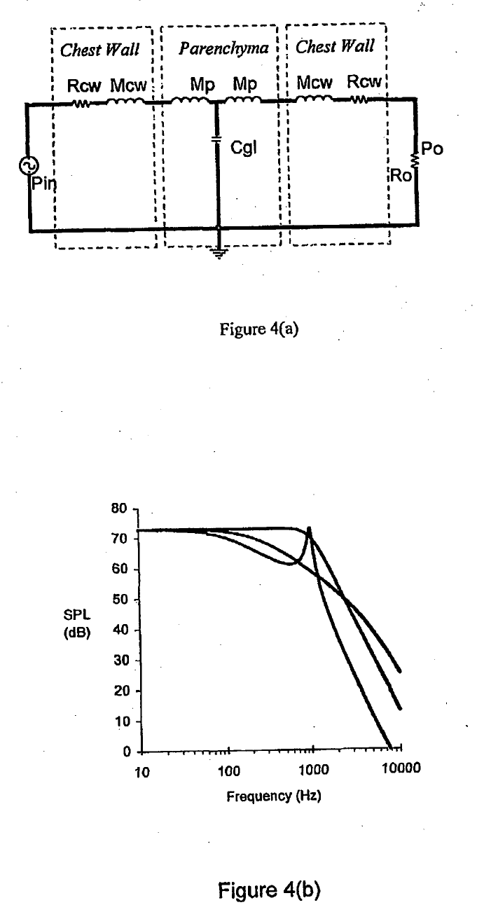

The essential features of this model as it relates to the thorax can be represented by an electrical equivalent circuit that can be used to describe the pertinent features of sound transmission through the thorax. This model is illustrated in FIG. 4(a). This approach to the analysis of acoustic transmission across the thorax facilitates analysis using sophisticated circuit emulation software such as SPICE to explore the effect of changing model parameters. In the equivalent electric circuit model where:
R_{cw} is the loss component associated with the chest wall and parenchyma;
M_{cw}, Mₚ is the surface mass of the chest wall and parenchyma respectively;
C_{gl} is the lung gas compliance;
Pᵢₙ, Pₒ are the acoustic input and output sound pressure levels respectively; and
   R₀ is the acoustic impedance of free space (414 MKS Rayls).

As illustrated in FIG. 4(b), the model can be used to simulate the effect that changing R_{cw} has on the transfer function of the equivalent circuit which represents the chest. This transfer function can be described mathematically as Pₒ(f)/Pᵢₙ(f) where f is the frequency and Pᵢₙ(f) and Pₒ(f) are the input (transducer) and output (chest microphone) sound pressure levels (SPL) respectively. As R_{cw} is decreased, the transfer function becomes progressively more peaked or resonant as illustrated by curves 1 to 3 in FIG. 4(b).

At sufficiently high frequencies, the output sound pressure level for all three curves falls asymptotically at a rate of 60 dB per decade. As the frequency is increased above the resonant frequency, the response is dominated by the inertial mass of the proximal and distal chest walls, and the shunt gas compliance of the lung. These act together to produce the 60 dB per decade fall-off, such that the thorax is, in effect, acting like a third order low-pass electrical filter. Analysis of the equivalent circuit, neglecting losses, shows that the resonant frequency of the thorax, f₀, can be determined using:

$${f}_{0} = \frac{1}{2 π} \sqrt{\frac{2}{{C}_{gl} ( {M}_{cw} + {M}_{p} )}} . \qquad ( 8 )$$

Furthermore, if the transfer function is measured at f₀ and at another frequency well above f₀, say, 3f₀ then using an analysis of the equivalent circuit, an explicit expression for lung gas compliance, C_{gl}, can be deduced in the form

$${C}_{gl} = \frac{4.18 \times {10}^{- 2} G}{{f}_{0}} , \qquad ( 9 )$$

where G=|Pₒ(f)/Pᵢₙ(f)| and is the magnitude of the transfer function of the thorax measured at 3f₀. This equation has been verified using SPICE simulation.

It follows that gas volume V_{gl} can be computed using equation 10:
V_{gl}=γP₀C_{gl} (10)
where γ is the adiabatic gas constant and P₀ is the atmospheric pressure.

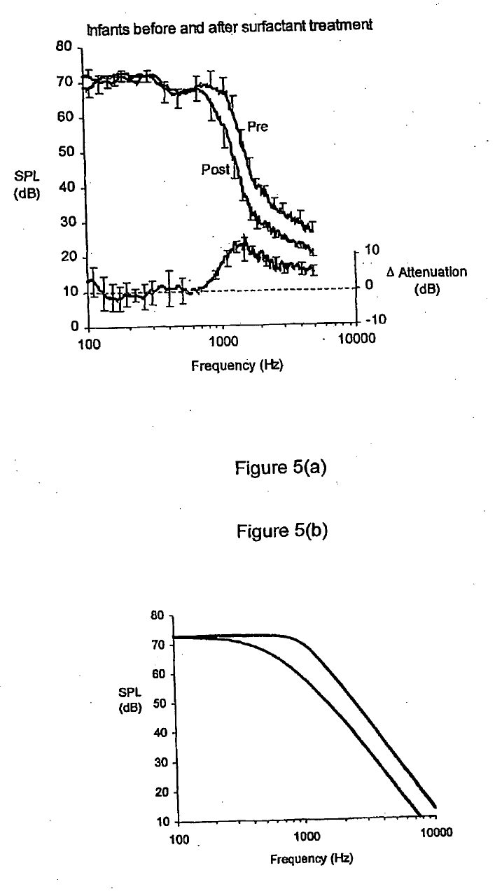

A further important application of this model is illustrated in FIGS. 5(a) and 5(b). FIG. 5(a) shows the experimentally measured thorax transfer function in a preterm infant soon after delivery but before surfactant administration (pre) and after the administration of surfactant (post) (Sheridan 2000). There is a steep fall-off in sound transmission for frequencies above 1000 Hz pre-surfactant and the leftward shift of this fall-off accompanied by an increase in attenuation of 10 dB following surfactant administration. A similar 10 dB change can be simulated in the model by increasing C_{gl} by about a factor of three while maintaining other parameters constant as illustrated in FIG. 5(b). Although a measurement of lung gas compliance was not made during these experiments, and is not feasible using currently available technology, it would be expected that such an increase in compliance (associated with an increase in gas volume) would occur after surfactant administration.

An important component of acoustic transmission which can be modeled using the equivalent electric circuit is the loss component R_{cw} illustrated in FIG. 4(a) which includes acoustic loss in the chest wall and parenchyma. Because the chest wall is acoustically thin, the dissipative loss in the wall is negligible but the loss in the parenchyma, which includes a large number of serial mass-compliance interfaces formed from the tissue and gas comprising the parenchymal structure, may be considerable. One model that has been proposed to account for acoustic loss in the parenchyma comprises air bubbles in water, for which an analysis already exists. In this model, absorption occurs because acoustic work is required to alternately compress and expand these bubbles.

It has been shown (Wodicka 1989) that the plane wave attenuation produced by N bubbles over distance x is given by:

$$P ( x ) = {P}_{0} {ⅇ}^{- ( \frac{N σ}{2} ) x} , \qquad ( 11 )$$

where
σ=16π²rₒ⁴ρₜcₜ R/{R²+(ωM−1/ωC)²};
P(x) is the SPL at x;
Pₒ is the SPL at x=0;
r₀ bubble radius;
cₜ sound speed in tissue; and
R,M,C are the effective mechanical resistance, mass and compliance of the bubbles respectively.

Attenuation,

$$α = \frac{P ( x )}{{P}_{0}}$$

in dB/cm can then be written as:
α=4.35Nσ (12).

This is a complex function of R,M,C but a simplified expression for the attenuation can be deduced by recognising that the acoustic vibration of the bubbles (alveoli) is dominated by bubble compliance C at frequencies which are much lower than resonance (ie. <≈10 kHz for realistic alveoli sizes). Therefore, attenuation can be reduced to:
α=2.36×10⁻²r₀⁶f³N (13).

The number of bubbles per unit volume N is approximately related to the gas fraction (1−h) by:

$$N = \frac{3 ( 1 - h )}{4 π {r}_{0}^{3}} , \qquad ( 14 )$$

hence equation 13 can be written as

$$α = 1.35 \times {10}^{- 3} \frac{{{f}^{3} ( 1 - h )}^{2}}{N} . \qquad ( 15 )$$

From these equations, it can be seen that:

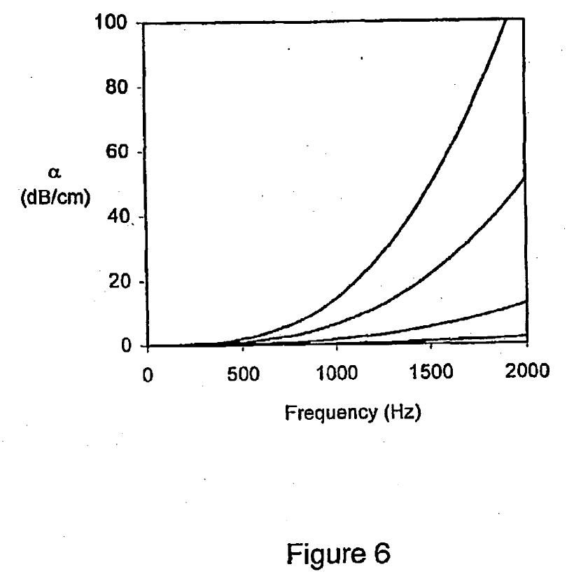

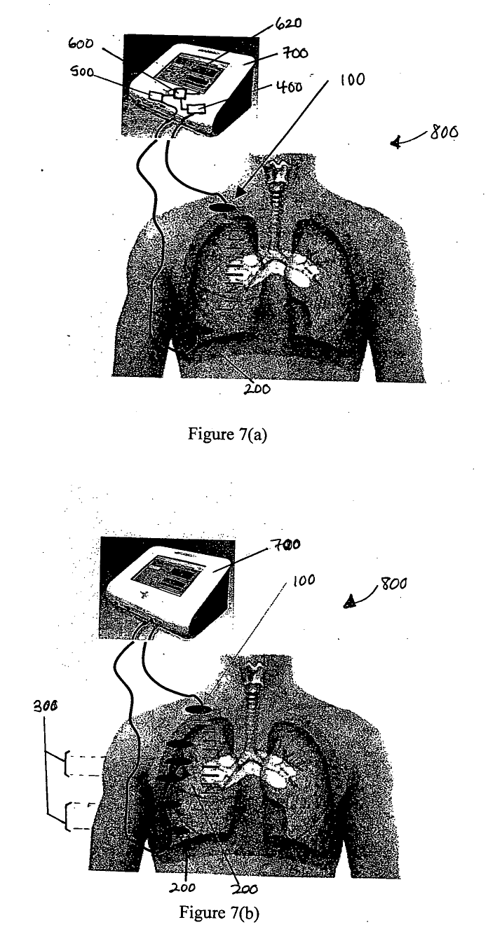

(a) absorption is related to the square of the gas fraction (1−h); a small increase in the tissue fraction h is associated with a marked decrease in high frequency attenuation (FIG. 6). This may explain the increased transmission of sound across the chest wall which can be observed clinically at high frequencies, following pneumonic consolidation of the lung; and

(b) attenuation is a strong function of both the frequency f and the alveolar radius r₀. This may explain, in part, the rapid fall-off in transmitted sound at high frequencies seen in both adult and neonatal subjects. The dependence on bubble radius may explain the reduced transmission through the thorax during emphysema.

Furthermore, these equations indicate that:

(a) absorption is related to the square of the gas fraction (1−h); and

(b) sound transmission attenuation is a strong function of both the frequency and the alveolar radius.

Using these relationships between sound transmission velocity in tissues and the tissue characteristics themselves, it is possible to obtain a workable relationship between acoustic measurements and lung pathology or the pathology or condition of other biological tissues.

This method provides a virtually continuous real-time measurement of tissue characteristics by analysing the velocity and attenuation of a defined sound as it propagates through the tissue. The method is applicable in both adults and infants, and for humans and animals. In particular, the present invention can be used in the determination of respiratory conditions in infants who cannot co-operate with presently available conventional stethoscopic methods of respiratory condition analysis which requires vocal co-operation. It is also useful where the patient is critically ill, is unconscious, or is unable to respond or generate a sound which can be used to determine lung condition.

In a preferred aspect of the present invention, there is provided a method of determining a state of the upper airways in a respiratory tract in a patient in situ, said method including:

introducing a sound at a first position in the upper airways;

detecting the sound after it has travelled through the upper airways at another position spaced from the first position;

calculating the velocity and attenuation of the sound travelled through the upper airways from the first position to another position; and

correlating the velocity and attenuation of the sound to the state of the upper airways.

The state of the upper airways may include any condition of the upper airways such as obstructed or open airways. Measurement of the closure or collapse of the upper airway is particularly useful for conditions such as in obstructive sleep apnea or OSA.

Apnea, and particularly Obstructive Sleep Apnea (OSA) is associated with closure of the upper airway and lapses in respiration during sleep. Using the present invention, a pseudo-random noise may be introduced into the airway using an acoustic transducer which conducts the sound from a location in the upper airway preferably via a Silastic nosepiece adapter. During normal respiration, the airway is open and the sound is transmitted via the airway to the lung via the trachea, where it subsequently propagates through the lung parenchyma and thorax to the surface of the chest. A sound-detection device such as a microphone may be attached in the chest region. Variations in the sound level which is measured at the chest region can then be used to model the degree of upper airway patency. The chest region may include the region extending from below the buccal cavity to below the lung. In the case of a baby or an infant, the sensor may be placed on the chest.

Preferably, the microphone is placed on the upper chest region generally below the neck and just above the lung.

When the airway is closed, the transmission of sound through the tissue decreases so that it may be undetectable by a microphone located on the chest. Therefore, when the sound falls below a certain value, it is likely to indicate the closure of the airway. When the signal that is detected by a microphone detector located on the chest region falls below a certain preset limit, an alarm is activated indicating obstruction of the airway. This alarm may wake up the subject which will most often result in the subsequent reopening of the airway, or it may alert attending staff to a patient who is being monitored for OSA or any other airway dysfunction. There are several benefits associated with this method for detecting airway obstruction or closure which include:

(a) the technique is non-invasive;

(b) the technique can be used in new-borns and adults alike, and in humans or in animals; and

(c) the technique monitors patency of the airway, not depletion of oxygen or lack of movement as is the case in other apnea detection devices. As a result of this, the susceptibility of the subject to oxygen depletion is detected before depletion itself occurs, reducing the likelihood of discomfort and tissue damage which can be caused by extended lapses or pauses in regular respiration and oxygen deprivation. This method can also be used to set the optimal level of CPAP to apply to a patient in order to maintain airway patency.

In yet another preferred aspect of the present invention, there is provided a method of monitoring lung condition in situ said method comprising:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

correlating the attenuation and sound velocity and velocity dispersion to lung condition.

Previous work shows that measurement of sound velocity alone may provide a technique for assessing lung density and gives an insight into the degree of lung inflation. However, no attempt has been made to evaluate the potential utility of sound velocity and attenuation as a clinical tool.

Lung condition may be selected from the group including but is not limited to:
(a) lung tissue density;
(b) lung gas volume;
(c) regional collapse (atelectasis);
(d) regional blood volume, interstitial oedema; and
(e) focal lung pathology such as tumour and global lung disease such as emphysema.

The lung conditions may then be compared with the condition of a normal, healthy lung.

To measure lung condition, the method of the present invention is preferably applied by introducing a sound to the thorax and hence to the lung preferably by applying an acoustic transducer to the thorax on one side of the chest and calculating the sound velocity and attenuation using a detector or microphone which is attached to the other side of the chest and which detects the transmitted sound. Previous measurements of lung condition or volume have been made by introducing sound to the lung tissue via the trachea. However, there are problems associated with this method for the lung which result from the unknown distance between the trachea and chest wall, and an inability to selectively distinguish the effects of the airway from the effects of the lung parenchyma on the velocity of the introduced sound. In other measurement techniques, the sound is generated by the subject by respiration, coughing or speech, or is introduced through percussion. However, this presents a key limitation because the acoustic properties of these sounds are subject-dependent and beyond control, particularly in the newborn infant, who is unable to reliably produce the desired sound on command.

The present invention exhibits a novel approach to examining the acoustic properties of the biological tissues, including the upper airways and of the thorax, by introducing sounds with a known and precisely defined spectral content as the investigative tool. For the lung, by utilising this sound which is introduced directly to the wall of the thorax, and by recording the sound after it is transmitted across the thorax, uncertainties associated with noise introduced via the trachea are eliminated. Without being restricted by the theory, research suggests that the lung tissue type which is primarily responsible for changes in sound velocity as it propagates through the thorax is the lung parenchyma; the contribution to changes in sound wave velocity and attenuation which is made by the airways is insignificant.

Many lung diseases are associated with characteristic features that can be detected using auscultation of the chest (Lowe and Robinson, 1970). In the normal lung, frequencies above 300-400 Hz are heavily attenuated by thoracic tissue, and on auscultation, respiration sounds are soft, conversational sounds are muffled, and whispered sounds are inaudible. By contrast, pneumonic consolidation greatly reduces the attenuation of high frequency sounds, resulting in characteristic respiration sounds known as ‘bronchial breathing’ and strong transmission of whispered (high-frequency) sounds known as ‘whispering pectriloquy’. A pleural effusion on the other hand, classically gives rise to increased attenuation of low frequency sound, causing vocal sounds to have a high pitched nasal quality known as ‘aegophony’.

Studies have been published which examine the effect of lung condition on sound attenuation in the healthy human lung. However, these studies have failed to measure the effect of lung inflation on sound attenuation. The present invention utilises transthoracically introduced sound and preferably measures the sound velocity and sound attenuation to determine lung condition. Lung conditions assessed using the present invention may include lung density and lung volume. However, other lung conditions may be determined by correlating changes in sound velocity and sound attenuation which are associated with known lung conditions with sound velocities and attenuation which are measured using a normal, healthy lung.

Tissue density may be measured using sound velocity alone. However, sound attenuation may also be introduced as a parameter for the determination of tissue density. Tissue density may be a measure of the amount of fluid or blood in the tissue. In the lung, it may also indicate gas volume, regional collapse (atelectasis), regional blood volume, interstitial oedema and both focal lung disease (eg tumour) and global lung disease (eg emphysema) which may be compared with a normal, healthy lung.

In yet another preferred aspect of the present invention there is provided a method of measuring lung inflation, said method including:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

correlating changes in sound velocity and attenuation with lung volume and inflation.

Lung gas volume is inversely proportional to lung density and may be measured using sound velocity and preferably sound attenuation. Furthermore, measurement of the velocity of a sound as it propagates from one side of the thorax through the lung tissue to the other side of the thorax can be correlated with a change in lung volume (inflation). This may be done in isolation, or during or after clinical interventions which alter the degree of lung inflation.

Measurements taken may include:
a) before and at intervals after treatment with surfactant;
b) before and at intervals after commencement of Continuous Positive Airway Pressure (CPAP) to recruit lung volume in the presence of hyaline membrane disease and/or atelectasis;
c) before and at intervals after the commencement of mechanical ventilation; and
d) before and immediately after endo-tracheal tube suctioning.

The degree of change in the sound velocity and preferably also of sound attenuation may be used together to provide a more conclusive indication of the degree to which the lung is inflated. Lung inflation may be determined using a single measurement, or it may be determined continuously, thereby enabling the monitoring of progress of lung disease and its treatment. This has particular value in the treatment and monitoring of lung disorders in premature babies over a period of time.

In yet another preferred aspect of the present invention, there is provided a method of predicting chronic lung disease in infants said method including:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

comparing the measured sound velocity and attenuation with that of a normal lung in the absence of chronic lung disease.

Abnormal lung density due to over- or under-inflation of the lung may be associated with increased lung injury and the propensity for development of chronic lung disease in infants. Therefore, measurements of sound velocity and attenuation (which relate to lung density) in a premature infant may allow inflation to be optimised and risk of chronic lung disease to be reduced.

Measurements of the sound velocity and sound attenuation may be made on days 1, 2, 3, 5, 7, 10 and 14 or any interval thereof and then at weekly intervals until about 36 weeks. As a comparison, and to complement measurements made using the present invention, absolute lung volume may be measured using the gold-standard and long-established helium dilution technique at the time of the acoustic measurements. Results taken from infants who subsequently develop chronic lung disease (defined either as oxygen dependency at 28 days or at a postmenstrual age of 36 weeks) may be compared with results from those who do not.

In yet another preferred aspect of the invention there is provided a method of diagnosing lung disease, said method including measuring lung density including:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax; and

correlating sound velocity and attenuation with lung density and comparing the density of the lung being diagnosed with the density of a normal lung to determine if the lung being diagnosed is diseased.

A similar technique can be used to assist in diagnosing lung disease wherein again, a sound is introduced to the thorax such that it travels from one side of the thorax, through the lung, to another side of the thorax. The sound velocity and preferably attenuation which are measured are then compared with that of a normal, healthy lung. Since lung disease often manifests in reduced lung volume, a comparison can be used, again, to provide an indication as to whether a subject's lung exhibits a propensity for lung disease, Common lung diseases may include emphysema, asthma, regional collapse (atelectasis), interstitial oedema and both focal lung disease (e.g. tumour) and global lung disease (e.g. emphysema). Each of these may be detectable when measurements of the velocity and attenuation of a sound which is transmitted through a diseased lung are compared with those of a lung in normal condition.

In yet another preferred aspect of the present invention, there is provided a method of preventing lung injury, said method including monitoring lung condition by:

introducing an audible sound transthoracically so that the sound travels from one side of the thorax, through the lung, to another side of the thorax;

measuring the velocity and attenuation of the sound as it travels from one side of the thorax, through and across the lung, to the other side of the thorax;

correlating the sound velocity and attenuation with lung volume; and

maintaining a lung volume at an optimal volume such that the lung is substantially free of atelectasis or over-inflation (volutrauma).

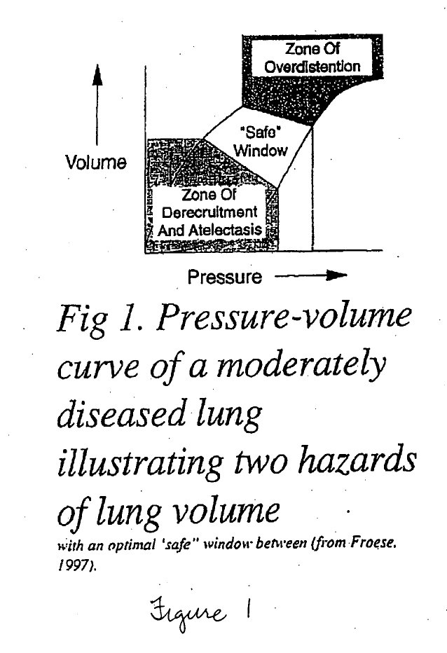

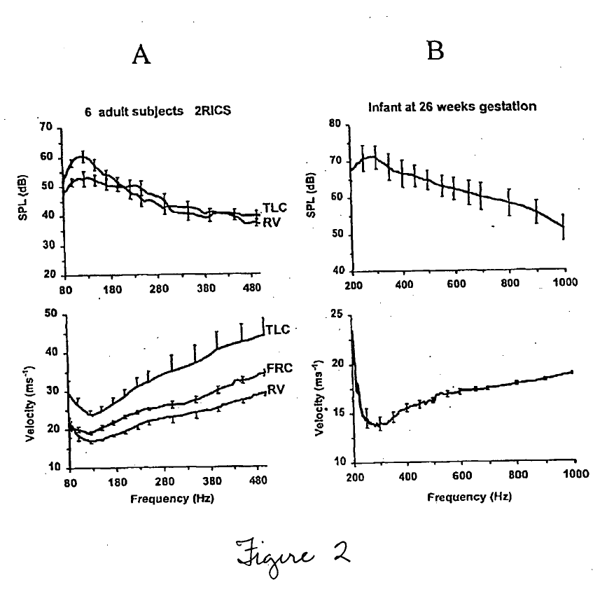

The present invention provides a reliable method for monitoring lung density and volume in situ. However, it can also be used to provide a method of preventing lung injury by again, introducing a sound transthoracically so that the sound travels from one side of the thorax through the lung to another side of the thorax. The velocity of the sound can be measured as it travels from one side of the thorax through the lung to the other side of the thorax, and the measurement can be used to indicate the volume of the lung which can then be used in the maintenance of an optimal lung volume which is substantially free of atelectasis or over-inflation (volutrauma). These optimal lung volumes are illustrated graphically in FIG. 1, wherein there exists a window inside which the possibility of causing lung injury can be minimised. This window is framed by under-inflation and over-inflation lung volumes. If lung volume is maintained inside this window, the likelihood of lung injury will be reduced. However, to ensure the volume does not rise excessively and does not drop to the level of atelectasis, it is necessary to constantly monitor the lung's volume.

In another aspect of the present invention there is provided an apparatus for determining characteristics of tissue from the respiratory system, the apparatus including:

a sound generating device which generates an audible sound;

a recording device which records the sound after it has travelled from one position of the biological tissue, through the tissue and to another position of the tissue; and

an analysis device which calculates the velocity and attenuation with which the sound travels through the tissue, and which can preferably perform spectral analysis on the data recorded.

In yet another preferred aspect of the present invention, there is provided an apparatus for monitoring lung condition, said apparatus including:

a sound generating means to generate an audible sound transthoracically so that the sound travels from one side of the thorax, through the lung, to another side of the thorax;

a recording means to record the sound after it has travelled from one side of the thorax, through and across the lung, to the other side of the thorax; and

an analysis device which calculates the attenuation and velocity with which the sound travels from one side of the thorax, through and across the lung, to the other side of the thorax, and which can preferably perform spectral analysis on the data recorded.

The present invention can be used to provide a monitoring system which measures sound velocity and preferably combines sound velocity data with measurements of sound attenuation in order to determine the level of lung inflation in a subject. Spectral analysis of the impulse response can indicate frequency components in the sound signal which are more prominent than others and which may be an indicator of pathological or abnormal tissue.

The benefits associated with the application and detection of acoustic signals to biological tissues is not limited to the lungs, airways and other tissues associated with respiration. The present invention can be used to detect densities of other porous structures and composite biological tissues which have high or low densities, wherein the ratio of solid to porous tissue gives rise to the change in velocity and sound attenuation which is measured.

The term “auscultation” is commonly used and well known in medical circles. Herein, the concept or technique commonly known simply as “auscultation” is referred to as “passive auscultation” in order to distinguish it from an “active auscultation” concept or technique associated with the present invention, as further described below.

The term “passive auscultation” generally refers to receiving at least one naturally occurring sound from the body or a portion thereof for use in the diagnosis and/or treatment of the body or a portion thereof. Passive auscultation may be limited in application for a number of reasons, such as those now described. For example, some parts of the body produce little or no natural sound, or natural sound that is difficult to receive or detect, such that passive auscultation is not particularly useful in connection with those parts of the body. Further by way of example, some of the sounds that can be received or detected by passive auscultation are not that much affected by the bodily matter they pass through, such that they are not particularly useful in diagnosis or treatment of that bodily matter. Still further by way of example, in passive auscultation, the condition of an original sound that may pass to or into the bodily matter is generally not known, such that it is difficult to adequately analyze, particularly in a quantitative manner, the relative nature or condition of the received sound.

The term “active auscultation” generally refers to actively introducing at least one first sound into the body or a portion thereof and thereafter receiving at least one second sound, such as a sound that is derivative or responsive relative to the first sound, from the body or a portion thereof for use in the diagnosis and/or treatment of the body or a portion thereof. The first sound that is introduced to the body or portion thereof may be selected to suit its particular application, such as a first sound that when so introduced is sufficient for producing a sufficiently discernible or detectable second derivative or responsive sound. The first sound that is introduced to the body or portion thereof may also be known in terms of any of various conditions, such as the time of the introduction of the sound or any of various parameters of the sound, such as the sound pressure level, the phase of the sound, the frequency of the sound, the velocity of the sound, and/or the like, for example, such that the relative nature or condition of the second derivative or responsive sound may be analyzed in a meaningful way, such as quantitatively, for example. Active auscultation is thus generally a more useful and powerful technique than passive auscultation.

The derivative or responsive second sound that is associated with active auscultation may be any sound that has been transmitted through a body or a portion thereof. It will be appreciated that a transmitted sound that has been transmitted through a body or a portion thereof may give rise to a second responsive sound according to any of a number of physical phenomena. By way of example, this second sound may comprise a transmitted sound that has been transmitted through a body or a portion of thereof, substantially without variation from the direction of the first sound. Further by way of example, this second sound may comprise a reflected sound that, after having been transmitted through a body or a portion thereof, has been at least partially reflected, such as in a general direction, such as a backward direction, that is opposite the general direction of the first sound, and at an angle relative to the direction of the first sound. Still further by way of example, this second sound may comprise a scattered sound that, after having been transmitted through a body or a portion of thereof, has been at least partially scattered, such as in a number of directions and angles relative to the direction of the first sound. Yet further by way of example, this second sound may comprise a refracted sound that, after having been transmitted through a body or a portion of thereof, has been at least partially refracted, such as in the same general direction as the first sound, such as in a forward direction, but at an angle relative to the direction of the first sound. Still further by way of example, the second sound may comprise any combination of sounds just described.

The derivative or responsive second sound may come from one or several locations. Further, multiple derivative or responsive second sounds may come from one or several locations. A device that is used to receive the second sound may be placed in any appropriate manner to receive the sound, as may be desirable or predicated by the nature of the second sound. Further, any useful combination of such devices, appropriately placed, may be used.

In general, active auscultation may involve the cross-correlation of the first sound that is introduced to the body or a portion thereof and the second sound that is received from the body, be it a transmitted sound, a reflected sound, a scattered sound, a refracted sound, and/or the like, and obtaining meaningful information from the correlation, such as a time delay or a phase shift, merely by way of example. The information obtained may concern a single parameter, such as a sound velocity, for example, multiple parameters, such as a sound velocity and a sound attenuation, for example, and/or a ratio of parameters, such as a ratio of a first sound velocity and a second sound velocity, for example, as further described herein.

According to the present invention, any of various parameters of the derivative or responsive sound may be received or determined. A consideration of a single sound parameter may be useful in assessing or determining a condition of a body or a portion of a body. Examples of such single sound parameters include an amplitude, a pressure, a velocity, a frequency, an attenuation, a phase, a time, and/or the like, associated with sound, any of which may indicate an absence and/or a presence of sound.

A consideration of multiple such parameters, particularly those received or determined substantially simultaneously, may be powerful in terms of determining at least one bodily characteristic, such as at least one condition of a portion of a body. According to an embodiment of the invention, active auscultation comprises using at least two parameters selected from any of the single parameters described herein. According to another embodiment of the invention, active auscultation comprises using at least two parameters selected from a velocity associated with the derivative sound, an attenuation associated with the derivative sound, and/or a frequency associated with the derivative sound. According to another embodiment of the invention, active auscultation comprises using at least one ratio selected from a ratio of a velocity of the first sound and a velocity of the second sound, a ratio of an attenuation of the first sound and an attenuation of the second sound, and/or a ratio of a frequency of the first sound and a frequency of the second sound. According to yet another embodiment of the invention, active auscultation comprises using at least one ratio of a first parameter selected from a velocity of the first sound, an attenuation of the first sound, and/or a frequency of the first sound, and a second parameter selected from a velocity of the second sound, an attenuation of the second sound, and/or a frequency of the second sound. According to still another embodiment of the invention, any useful combination of sounds from one receiving point, another receiving point, one set of receiving points, and/or another set of receiving points, may be used.

Merely by way of example, the parameters of sound velocity and sound frequency may be powerful in terms of determining at least one bodily characteristic, such as at least one condition of a portion of a body. Merely by way of example, in certain circumstances or cases, the effect of propagation of sound through a portion of a body on sound velocity may be most marked in relation to a sound frequency band or a sound frequency, and relatively less marked in relation to another sound frequency band or another sound frequency. In such a circumstance or case, active auscultation may involve a determination of sound velocity that is determinably affected, significantly affected, and/or most affected by propagation through a portion of the body, such as sound velocity at a sound frequency band or a sound frequency, as just described. Further, merely by way of example, in certain circumstances or cases, sound velocity at a particular sound frequency may change relatively slowly as a bodily condition (such as a disease condition, for example) changes, and sound dispersion (or a derivative of sound velocity as a function of sound frequency) may change relatively more rapidly. Such a circumstance or case may be that associated with a lung of an emphysematous subject, merely by way of example. In such a circumstance or case, active auscultation may involve a determination of sound velocity at each of two frequencies. Such a determination may allow for an estimation or a determination of sound dispersion, such as via determining a difference between the two sound velocities and a difference between the associated two sound frequencies, and dividing the former by the latter, merely by way of example.

Active auscultation may be used in connection with any suitable portion of a body, such as any portion of a body in connection with which active auscultation can provide meaningful information, such as information concerning a condition of that portion of the body, another portion of the body, and/or of the body itself, such as anything from a normal or healthy condition to an abnormal or unhealthy condition, merely by way of example. Examples of suitable portions of a body include those that are cavitary or cavernous, solid, fluid (such as liquid or gas), interstitial, vascular, muscular, skeletal, cardiac, cerebral, neural, pulmonary, respiratory, and any combination thereof, merely by way of example.

According to the present invention, there is at least one location for the introduction of sound and at least one location for the receipt of sound. Merely by way of example, sound may be introduced at one location and received at a number of locations. Sound may be introduced and received at the same or at different portions of the body, such as the same or different sides of the body, whether front, back, left, right, or any combination thereof. Preferably, a location for the introduction of sound and a location for receipt of sound do not interfere with the ability to receive a useful or meaningful sound or to process a received sound such that it is useful or meaningful.

Merely by way of example, the location for the introduction of sound may be of an upper airway of the respiratory system, such as a location associated with a nose or a mouth, and the location for the receipt of sound may be of another location of the upper airway, such as a location adjacent to (and preferably displaced somewhat from) the location for the introduction of sound, or a location associated with a neck or a tracheal region of the upper airway, by way of example. Such a configuration may be useful to ascertain a condition of an upper airway, such as whether the airway is open or obstructed, for example, as may be important in a variety of applications, such as monitoring for apnea and/or an obstruction or closure in the upper airway, for example. Merely by way of example, when sound is introduced to the upper airway via a nose and/or a mouth, a derivative sound that is transmitted to the other location of the upper airway and is reflected back to some point of the upper airway, such as the nose, mouth, and/or throat, for example, may be received via active auscultation. Generally, a derivative sound that is transmitted when the airway is unobstructed will be large in amplitude or strength relative to a derivative sound that is transmitted when the airway is obstructed. Generally, a derivative sound that is reflected back when the airway is unobstructed will be small in amplitude or strength relative to a derivative sound that is reflected back when the airway is obstructed. In this way, active auscultation may be used to assess a condition of the upper airway from fully open or unobstructed to partially obstructed to fully closed or obstructed.

Further by way of example, the location for the introduction of sound may be of an upper airway of the respiratory system, such as a nasal and/or an oral portion of the airway, and the location for the receipt of sound may be of another or lower location of the upper airway, such as a location associated with a neck and/or a tracheal region of the upper airway, or a location lower down in the airway, such as below a trachea and into a lung. As mentioned above, sound may be introduced in one location and received in a number of locations and sound may be introduced and received in the same or in different portions of the body, such as the same or different sides of the body, whether front, back, left, right, or any combination thereof. Merely by way of example, sound may be introduced to at least one location of the upper airway, such as a nasal and/or an oral area of the airway, and/or to at least one location of the thoracic area of the airway, on a front right side of the body, and received at a number of different locations of lower down the airway, such as below the trachea and/or the thorax, respectively, and/or into a lung on the front right side of the body. Such a configuration may be useful to ascertain a condition of a selected portion of the airway, such as any portion along the length of a lung. Detection and/or monitoring of such a condition may be carried out via active auscultation on the basis of sound transmission, reflection, scatter, and/or refraction, as described above.

Still further by way of example, the location for the introduction of sound may be of a middle airway, such as below a neck region and/or a tracheal region of the upper airway, and the location for the receipt of sound may be of a lower portion of the airway. An example of such a configuration is shown in FIG. 7(a), where at least one location 100 for the introduction of sound is of the middle airway, near the top of a lung and a collar bone of a subject, and at least one location 200 for the receipt of sound is of a lower portion of the airway, near the bottom of the lung and displaced from the center of the body, such as near an outer ribcage of the subject. In the apparatus 800 shown in FIG. 7(b), there are multiple locations 200 for the receipt of sound. Such a configuration may be useful to obtain information concerning one or more segment(s) 300 of the sound path relative to the original sound and/or relative to one another. Accordingly, such a configuration may be useful to obtain information concerning one or more portion(s) of the body independently or relative to one another.

As mentioned previously, according to an aspect of the present invention, a method may be directed to determining placement of a structure within an airway. Various applications of such a method, such as those mentioned previously, for example, will be understood. Merely by way of example, such a method may be used to determine a placement, such as a correct or incorrect placement, for example, of a medical device, such as an endo-tracheal tube, for example, within an airway. This may be useful in a medical application in which placement of a medical device, and/or the monitoring thereof, is contemplated, for example. Such a method may comprise introducing at least one audible sound to at least one first bodily location associated with an airway, such as an upper airway, for example, the at least one audible sound sufficient to travel through at least a portion of the body to produce at least one responsive sound; receiving the at least one responsive sound from at least one second bodily location, such as a location that is spaced from the first bodily location, for example; determining an attenuation associated with the at least one responsive sound; and determining a placement associated with a structure within the airway, such as via correlating the attenuation to a placement of the structure, for example. Such a method may be used for the detection of a misplaced endo-tracheal tube, for monitoring of a placement of an endo-tracheal tube, for on-going assessment of appropriate placement of an endo-tracheal tube, and/or the like. Merely by way of example, an endo-tracheal tube may be positioned sub-optimally such that it resides in either the left principal bronchus or the right principal bronchus. Such a positioning might result in a difference in attenuation associated with the left and right sides of the thorax. A response to such a difference in attenuation might be uneven treatment as to the left and right sides of the subject, which may result in an over-inflation and/or an under-inflation of the left and/or right lung(s).

Active auscultation may be carried out using an apparatus such as that shown in FIGS. 7(a) and 7(b). The apparatus 800 comprises at least one element 400 sufficient for producing an audible sound and communicating it to a location 100 for the introduction of the sound to the body or a portion thereof. The apparatus 800 comprises at least one element 500 sufficient for receiving a derivative or responsive sound from at least one location 200 for the receipt of sound. The apparatus 800 may further comprise at least one console 700 that may house an element 400; an element 500; a processor 600, such as a microprocessor, for example, sufficient for processing information obtained, whether information concerning an audible sound, such as information from element 400, for example, or information concerning a responsive sound, such as information from element 500, for example; and/or at least one element 620 sufficient for the communication of raw and/or processed information, which may take the form of at least one display, as shown, such as a display of numerical, textual, graphical, and/or representational information, and may have at least one alarm and/or other sensory notification capability. The apparatus 800 may further include at least one user interface (not shown), as may be provided in connection with a console 700, for the interaction of the user with the apparatus, such as for the input of data, for example.

The apparatus 800 and any element or component thereof, such as the microprocessor 600, may comprise any appropriate element(s) or component(s) for achieving any desirable or intended purpose(s). Examples of such element(s) or component(s) include any one or more of the following: electronic circuitry, componentry, storage media, signal- or data-processing element(s), algorithmic element(s), software element(s), logic device(s), wired or wireless communication element(s), device(s) for operable communication between elements or components, and the like. The microprocessor 600 may be configured to include any suitable element(s) described herein, or any suitable element(s) for achieving any of the purpose(s) described herein, in a conventional manner. Any device with which the microprocessor 600 may communicate may be equipped with complementary element(s), such as any suitable communication element(s), component(s), or device(s), such as wired or wireless communication element(s), merely by way of example, as may be afforded or accomplished in a conventional manner.

Active auscultation methods and apparatus may be used in connection with a medical process or a medical device. For example, a method or an apparatus of the invention may be used in the monitoring and/or the controlling of a medical device, for example. Merely by way of example, active auscultation may be used in connection with a ventilator, such as to control the ventilator based on the results of the active auscultation. For example, if active auscultation shows a lung to be over-inflated, under-inflated, and/or otherwise in an undesirable air-fill condition, that information may be used to provide notice of such a condition so that a person may adjust the ventilator accordingly, and/or may be used in a feedback control loop that automatically adjusts the ventilator accordingly. Such a technique or system may be useful in connection with the maintenance of desirable lung inflation and/or deflation, the optimization of lung inflation and/or deflation, the avoidance of chronic lung disease, the minimization of the likelihood of chronic lung disease, the treatment of chronic lung disease, and/or the like, merely by way of example.

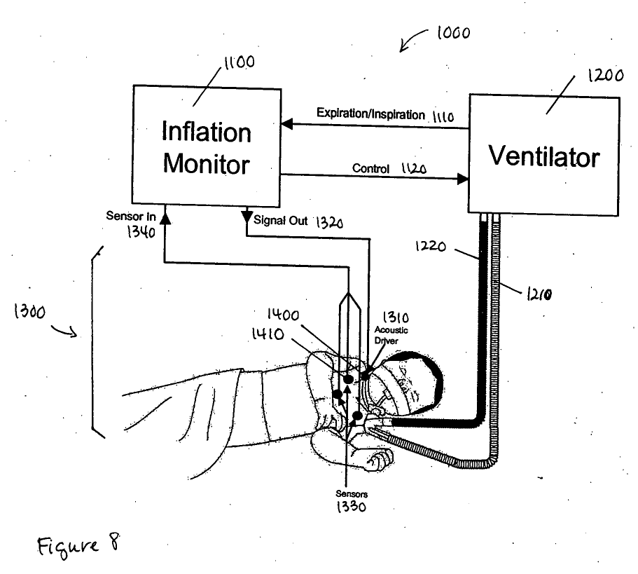

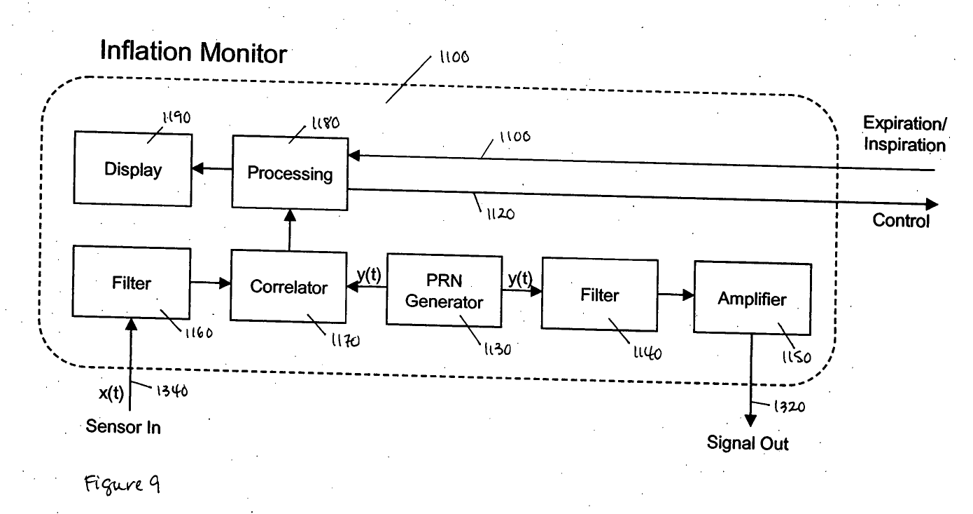

An example concerning the use of an active auscultation method and apparatus in connection with a medical process or a medical device is now described with reference to FIGS. 8 and 9. As shown in FIG. 8, a system 1000 may comprise an inflation monitor 1100 and a ventilator 1200 in operable communication with one another. The inflation monitor 1100 is sufficient for monitoring a respiratory condition (inspiratory (lung inflation) and/or expiratory (lung deflation)) associated with the ventilator 1200, which monitoring may be intermittent or continuous. The ventilator 1200 is sufficient for operable communication with a subject, such as a human infant as shown, for example, via a channel 1210 sufficient for supplying inspiratory gas, such as air, to the subject and a channel 1220 sufficient for removing expiratory gas, such as carbon dioxide, from the subject. The ventilator and the respiratory channels may be of any suitable configuration and operation, as known. The inflation monitor 1100 is operably associated with the ventilator 1200 to receive information concerning a respiratory condition and to control the ventilator, as indicated schematically in FIG. 8 by the directional arrows 1110 and 1120, respectively, between the inflation monitor 1100 and ventilator 1200. Information concerning the respiratory phases associated with the ventilator 1200 may be provided via the ventilator 1200 itself, or may be otherwise obtained, such as via monitoring of pressure associated with operation of the ventilator 1200. The control of the ventilator may be based on such information and/or information from an active auscultation method and apparatus 1300, as now further described.

The active auscultation apparatus 1300 may comprise at least one transducer 1310, such as an acoustic driver, that is disposed relative to the subject at a location 1400 sufficient for the introduction of sound to the subject, such as via a signal output from the inflation monitor 1100, as indicated schematically in FIG. 8 by the directional arrow 1320 between the inflation monitor 1100 and the transducer 1310. The location 1400 may be on a surface of the subject in a suitable area for an application, such as a location associated with the upper airway of the subject, for example. The transducer 1310 is sufficient for introduction of sound to the subject at location 1400. The apparatus 1300 may further comprise at least one sensor 1330 that is disposed relative to the subject at a location 1410 sufficient for the receipt of a derivative or responsive sound that has passed though the thorax, for example, as previously described. The location 1410 may be on a surface of the subject in the area of interest, such as in the area of a lung of the subject. The sensor 1330 may be sufficient for monitoring a change in attenuation, a change in velocity, and/or a direction of a change in velocity associated with the derivative or responsive sound, as may be monitored intermittently or continuously throughout a respiratory cycle of the ventilator 1200. The inflation monitor 1100 is sufficient for receiving information from the sensor 1330, such as via a signal input to the inflation monitor 1100, as indicated schematically in FIG. 8 by the directional arrow 1340 between the inflation monitor 100 and the sensor 1330.

Generally, sound velocity in a lung that is already over-inflated, and may be at risk of volutrauma, increases to a relatively large extent when the lung is further inflated during an inspiratory phase of ventilation. A relatively smaller increase in sound velocity during an inspiratory phase of ventilation may generally be associated with a more optimal lung density or lung volume. Generally, sound velocity in a lung that is already under-inflated, and may be at risk of atelectasis, decreases to a relatively large extent when the lung is further inflated during an inspiratory phase of ventilation. Thus, a relatively large decrease in sound velocity during inflation may generally indicate that a lung is under-inflated, or of abnormally high density. Generally, when a lung is in a condition (such as of a lung density within a lung density range, for example) such that little change in sound velocity occurs upon inflation of the lung, attenuation may be used as an indicator of lung density or as a provider of information concerning lung density. As mentioned previously, the inflation monitor 1100 may be sufficient for controlling the ventilator 1200, such as adjusting parameters or settings associated with ventilation via the ventilator 1200, according to information available to the inflation monitor 1100. The inflation monitor 1100 may comprise any element(s) or component(s) as previously described in connection with the processor 600 associated with the apparatus 800 of FIGS. 7(a) and 7(b), such as a communication element for the communication of a respiratory condition, an alarm, and/or the like, to a user who may then adjust the ventilator 1200. The active auscultation apparatus 1300 may comprise multiple sensors 1330, such that regional over- and/or under-inflation may be identified via information obtained therefrom, and appropriate measures may be taken to address such a condition.

As just described, an inflation monitor 1100, such as that associated with a system 1000 of FIG. 8, for example, may comprise any of a number of element(s) or component(s). Merely by way of example, an inflation monitor 1100 and various elements or components thereof are schematically illustrated in FIG. 9, according to an embodiment of the invention. As shown, the inflation monitor 1100 may comprise an element 1130 for generating an audible sound, such as a pseudo-random noise, for example; an element 1140 for filtering the sound generated by the element 1130, such as a band pass filter, for example; and an element 1150 for amplifying the filtered sound from the element 1140, which amplified sound may then be provided as an output signal from the inflation monitor 1100, such as an output signal that may be communicated via a communication pathway 1320 to a transducer 1310, as previously described in relation to the system 1000 of FIG. 8, for example. As also shown, the inflation monitor 1100 may comprise an element 1160 for receiving an input signal, such as a band-pass filter sufficient for receiving an input signal that may be communicated via a communication pathway 1340 from a sensor 1330, as previously described in relation to the system 1000 of FIG. 8, for example. The element 1160 may comprise various element(s) or component(s), such as a power supply (not shown) for one or more sensor(s) 1330 and/or elements sufficient for signal conditioning, such as amplification, for example. As further shown, the inflation monitor 1100 may comprise a correlator for cross-correlating an input signal, such as that associated with a derivative or responsive sound transmitted through the thorax, x(t), for example, and a reference signal, such as that associated with an audible sound, y(t), from the generator element 1130. As still further shown, the inflation monitor 1100 may comprise an element 1180 for processing information from the correlator element 1170, and/or information from a ventilator 1200, such as information communicated via a communication pathway 1110 to the element 1180, for example. The processor element 1180 may be sufficient for communicating with the ventilator 1200, as previously described, such as to control operation of the ventilator, for example, based on information just described, information from a user (as may be provided via a user interface (not shown), for example), and/or the like, via an output signal that may be communicated via a communication pathway 1120 to the ventilator 1200. As also shown, the inflation monitor 1100 may also comprise an element 1190 for the display and/or communication of information, such as numerical, textual, graphical, and/or representational information, as may be of interest, useful, and/or desirable.

Examples relating to the present invention are now described.

### EXAMPLES

### Example 1

### Measurement of Lung Volume in Adults

In 6 healthy adult subjects, the velocity and attenuation of sound which was transmitted from one side of the chest to another, in a range of frequencies from 50-1000 Hz was measured at a number of defined positions on the chest. These measurements were taken while the lung volume was varied between Residual Volume (RV) and Total Lung Capacity (TLC). A reference position was established over the right upper zone of the chest. Using this position, a region in the frequency spectrum (around 100-125 Hz) where sound attenuation was much reduced and where the degree of attenuation was directly related to lung inflation (see FIG. 2, upper portion of panel A) was found. The difference in attenuation between RV and TLC was approximately 7.5 dB and statistically significant (P=0.028). Further, it was found that sound velocity was low, averaging around 30 m/sec, and it showed a clear and strong sensitivity to the degree of lung inflation, being appreciably faster at TLC than at RV (FIG. 2, lower portion of panel A). In this study evidence was found which indicated that the effect of inflation on velocity and attenuation varies at different locations in the thorax, particularly in the lower zones. It is likely that this is, in part, attributable to the location of the heart and liver (at RV) in the sound path.

The method of analysis permits determination of phase shift, and therefore velocity as a function of frequency. This work has shown that the speed of sound in the lung parenchyma is dispersive, or frequency dependent, over the range of frequencies studied. This is of considerable importance, since it is theorised that the relationship between velocity and frequency is dependent on regional compliance and inertial (ie mass dependent) properties of the lung. These properties may provide valuable information about the lung since they are partly determined by the condition of the alveolar septum, the degree of fluid infiltration of the lung parenchyma, and the extent of atelectasis.

Preliminary pilot data were collected from newborn infants in the neonatal intensive care unit. FIG. 2(b) represents a sample result from an infant of 26 wks gestation with healthy lungs, illustrating that measurements can be made using the present invention with a subject who cannot co-operate and who must be studied in the noisy intensive care setting. Interestingly, the frequency region over which sound attenuation is least in the newborn is higher (approximately 300 Hz) than in the adult. In addition, although the relationship between velocity and frequency has a nadir at about 300 Hz compared with 125 Hz in the adult, the dispersive nature of sound velocity which is evident in the adult is also present in the infant.

### Example 2

### Measurement of Lung Density in Rabbits

Experiments were conducted in 1-2 kg New Zealand white rabbits. These animals were chosen for their similarity in size to the human newborn and their widespread use as a model of neonatal surfactant deficiency. Animals were anaesthetised with intravenous thiopentone, before performing a tracheostomy during which a 3 mm endo-tracheal tube was inserted into the airway to allow ventilation using a conventional neonatal ventilator (Boumes BP200). Maintenance anaesthesia was achieved with intravenous fentanyl. The chest was shaved and a microphone and transducer secured in various pre-defined positions, including a reference position over the right upper chest. The animal was then placed in a whole body plethysmograph to monitor absolute lung gas volume at intervals throughout the experiment. Tidal volume was monitored continuously with a pneumotachograph attached to the tracheostomy tube. The sound velocity and attenuation was determined at each location of the where a microphone was situated, and each observation was the average of 10 repeated measures.

The effect of changes in lung density as a result of lung disease on sound velocity and attenuation was examined by comparing results from 3 groups of rabbits with differing lung conditions:

Group 1—Normal lungs (n=10);

Group 2—Lungs rendered surfactant deficient by saline lavage (n=10); and

Group 3—Lungs rendered oedematous by inflation of a left atrial balloon catheter (n=10).

Within each group of animals the effect of changes in lung density, resulting from changes in degree of lung inflation, was examined by making measurements under dynamic and static conditions.

(1) Dynamic measurements during mechanical ventilation. Sound velocity and attenuation may be measured during mechanical ventilation at various levels of positive end-expiratory pressure (PEEP) including 0, 5, 10, 15 and 20 cmH₂O. Absolute lung volume at end expiration, and tidal volume may be determined for each level of PEEP. A wide range of PEEP can be employed to ensure that observations are made over a wide range of lung volumes, from under-inflation to over-inflation and including optimal inflation.

(2) Static measurements during apnea. Sound velocity and attenuation was measured while the lung was transiently held at constant volume after spontaneous respiratory effort had been suppressed by a brief period of hyperventilation. Various lung volumes from below functional residual capacity (FRC) to TLC were achieved by varying airway pressure between −10 and +30 cmH₂O. Studying the lung under static conditions allows observations to be made at the extremes of lung volume. These results were directly comparable to observations during breath-hold in adult subjects and enables verification of the cross-correlation technique used in the present invention which increases the system's robusticity against interference from breath sounds.

(3) Static measurements post-mortem. At the completion of (2) above, a lethal dose of anaesthetic was administered and observations of sound velocity and attenuation were repeated across the same range of lung volumes as in (2). The trachea was then clamped at an inflation pressure of 10 cmH₂O before dissecting the lungs so that they were free from the chest and so that their weight and density could be determined. In order to address the question of the regional differences in sound velocity and attenuation observed in the adult human study, final measurements were made of the acoustic properties of the excised lung at the same levels as those studied in the intact thorax. An important aspect of this analysis is that it allowed comparison of results obtained before and after death to establish whether the cross-correlation technique used is resistant to interference from cardiac sounds.

### Example 3

### Measurement of Lung Inflation in Infants

To be a valuable clinical tool, measurements of sound velocity and attenuation must be sensitive to changes in lung inflation that are of a clinically relevant magnitude. A test of whether measurable changes in sound velocity and attenuation which occurred after clinical interventions which were confidently predicted alter the degree of lung inflation was conducted. It was found that clinical interventions which cause a significant change in lung inflation are associated with changes in sound transmission and velocity which are measurable using the present invention.

### Example 4

### Prediction of Chronic Lung Disease

It is also necessary to determine whether evidence from acoustic measurements of abnormal lung density are indicative of either under-inflation or over-inflation, and associated with development of chronic lung disease as a result. It was found that abnormal lung density in the first few days of life was more common in infants who subsequently developed chronic lung disease than in those who did not. Serial measurements of sound velocity and attenuation in a population of pre-term infants (n=30) who, by virtue of their gestation (<30 weeks), are at high risk of developing chronic lung disease were made. In this population and using the present invention, it was estimated that about 65% of the population will still be oxygen dependent at 28 days of age, and about 30% will still be oxygen dependent at a postmenstrual age of 36 weeks.

References, some of which may have been referred to previously in abbreviated form, are set forth below.

### REFERENCES

Australian and New Zealand Neonatal Network. Annual Report, 1996-1997.
Baumer J H. International randomised controlled trial of patient triggered ventilation in neonatal respiratory distress syndrome. Arch Dis Child 82: F5-F10, 2000.
Bernstein G, Mannino F L, Heldt G P, Callahan J D, Bull D H, Sola A, Ariagno R L, Hoffman G L,
Frantz I D 3^{rd}, Troche B I, Roberts J L, Dela Cruz T V, and Costa E. Randomized multicenter trial comparing synchronized and conventional intermittent mandatory ventilation in neonates. J Pediatr 128: 453-63, 1996.
Dreyfuss D, Basset G, Soler P and Saumon G. Intermittent positive-pressure hyperventilation with high inflation pressures produces pulmonary microvascular injury in rats. Am Rev Resp Dis 132: 880-884, 1985.
Fahy, F. (1985) Sound and Structural Vibration. Radiation, Transmission and Response. London: Academic Press.
Froese A B. Role of lung volume in lung injury: HFO in the atelectasis-prone lung. Acta Anaesthesiol Scand Suppl 90:126-130, 1989.
Froese A B. High frequency oscillatory ventilation for adult respiratory distress syndrome: Let's get it right this time! Crit Cae Med 25: 906-908, 1997
Gerstmann D R, Minton S D, Stoddard R A, Meredith K S, Monaco F, Bertrand J M, Battisti O, Langhendries J P, Francois A and Clark R H. The Provo multicenter early high-frequency oscillatory ventilation trial: improved pulmonary and clinical outcome in respiratory distress syndrome Pediatrics. 98: 1044-1057, 1996.
Goncharoff, V., Jacobs, J E, and Cugell, D W Wideband acoustic transmission of human lungs. Med. Biol. Eng. Comp. 27:513-519, 1989.
HIFI Study Group. High frequency oscillatory ventilation compared with conventional mechanical ventilation in the management of respiratory failure in preterm infants. N Engl J Med 320: 88-93, 1989.
Jobe A. Pulmonary surfactant therapy. N Engl J Med 328: 861-864, 1993.
Kraman, S. S. Speed of low-frequency sound through lungs of normal men. J. Appl. Physiol. 55:1862-1867, 1983.
Lowe R D and Robinson B F. A physiological approach to clinical methods. Churchill, London, 1970.
McCulloch, P R, Forkert P G and Froese A B. Lung volume maintenance prevents lung injury during high-frequency oscillatory ventilation in surfactant-deficient rabbits. Am Rev Respir Dis 137: 1185-1192, 1988.
Northway H Q, Rosen R C and Porter D Y. Pulmonary disease following respiratory therapy of hyaline membrane disease. N Engl J Med 276: 357-368, 1967.
Rice, D. A. (1983) Sound speed in pulmonary parenchyma. J. Appl. Physiol. 54:304-308.
Rife D D & Vanderkooy J. Transfer function measurement with maximum length sequences. J Audio Eng Soc 37: 419-444, 1989.
Sheridan, B (2000) Acoustic evaluation of lung inflation in the preterm infant. B. Med. Sci. Thesis, RCBHR, Monash University.
Taghizadeh A & Reynolds E O R. Pathogenesis of bronchopulmonary dysplasia following hyaline membrane disease. Am J Pathol 82: 241-264, 1976.
Wodicka, G. R. and Shannon, D. C. Transfer function of sound transmission in subglottal human respiratory system at low frequencies. J. Appl. Physiol. 69(6):2126-2130, 1990.
Wodicka G R, Stevens, K N, Golub, H L, Cravalho, E G and Shannon, D. C. A model of acoustic transmission in the respiratory system. IEEE Ttrans Biomed. Eng. 36: 925-934, 1989.

Various devices, systems, and methods have been described herein. It will be understood that a method of use or application is naturally contemplated in connection with any device or system described herein, and a device or system for carrying out a method is naturally contemplated in connection with any method described herein. It will be appreciated that each of the device and the method of the present invention may have many useful medical applications, as well a wide variety of other useful applications that involve the passage of audible sound at least partially through a medium and receipt of a responsive sound.

It is to be understood that various other modification(s) and/or alteration(s) may be made without departing from the spirit of the present invention as outlined herein. For example, various modification(s), process(es), as well as numerous structure(s) to which the present invention may be applicable will be readily apparent to those of skill in the art to which the present invention is directed, upon review of the specification. Various aspect(s) and/or feature(s) of the present invention may have been explained or described herein in relation to understanding(s), belief(s), theory(ies), underlying assumption(s), and/or working or prophetic example(s), although it will be understood that the invention is not bound to any particular understanding, belief, theory, underlying assumption, and/or working or prophetic example. Although the various aspect(s) and feature(s) of the present invention may have been described with respect to various embodiment(s) and specific example(s) herein, it will be understood that the invention is entitled to protection within the full scope of the appended claim(s).

1. A method of assessing at least one bodily characteristic, comprising:
introducing at least one audible sound to at least one first bodily location, the at least one audible sound comprising at least one known parameter and being sufficient to travel through at least a portion of the body to produce at least one responsive sound;
receiving the at least one responsive sound from at least one second bodily location;
determining at least two determined parameters associated with the at least one responsive sound, the at least two parameters comprising velocity and at least one other parameter selected from attenuation and/or frequency; and
assessing at least one characteristic of at least a portion of the body based on the at least one known parameter and the at least two determined parameters.

2. The method of claim 1, wherein said introducing is via percussion or via a transducer sufficient to produce the at least one audible sound.

3. The method of claim 1, wherein the at least one audible sound is sufficient to be distinguished from environmental noise present during said introducing.

4. The method of claim 1, wherein the at least one audible sound is selected from a tone, a sinusoidal wave, and/or a pseudo-random noise.

5. The method of claim 1, wherein the at least one known parameter of the at least one audible sound is selected from an amplitude, a pressure, a velocity, a frequency, a phase, and/or a time.

6. The method of claim 1, wherein the at least one characteristic of at least a portion of the body is selected from make-up, volume, condition, and/or position.

7. The method of claim 1, wherein the at least a portion of the body is selected from an airway, a thorax, and/or a lung.

8. An apparatus for use in assessing at least one bodily characteristic, comprising:
at least one transducer sufficient to provide at least one audible sound at at least one first bodily location;
at least one first detector sufficient to detect sound from at least one second bodily location and provide at least one first sound output;
at least one second detector sufficient to detect sound at at least one third bodily location and provide at least one second sound output;
at least one filter sufficient to remove very low frequency environmental noise from the at least one first sound output to provide at least one first filtered sound output and from the at least one second sound output to provide at least one second filtered sound output;
at least one amplifier sufficient to amplify the at least one first filtered sound output to provide at least one first amplified sound output and the at least one second filtered sound output to provide at least one second amplified sound output; and
at least one processor for processing the at least one first amplified sound output to provide at least one parameter associated with the at least one first sound output and the at least one second amplified sound output to provide at least two parameters associated with the at least one second sound output, the at least two parameters comprising a velocity and at least one other parameter selected from attenuation and/or frequency.

9. The apparatus of claim 8, wherein the at least one transducer is sufficient for attachment to a surface of the at least one first bodily location.

10. The apparatus of claim 8, wherein the at least one first detector is sufficient for placement on a surface of the at least one second bodily location.

11. The apparatus of claim 8, wherein the at least one detector is substantially in line with the at least one transducer and the at least one first detector.

12. The apparatus of claim 8, wherein the at least one processor is sufficient to perform a cross-correlation analysis based on the at least one first amplified sound output and the at least one second amplified sound output.

13. The apparatus of claim 8, further comprising a ventilator in operable communication with the at least one processor.

14. The apparatus of claim 13, wherein the at least one processor is sufficient for receiving respiratory information from the ventilator.

15. The apparatus of claim 13, wherein the at least one processor is sufficient for controlling the ventilator.
